(19) United States
(12) Patent Application Publication
(10) Pub. No.: US 2024/0327362 A1
(43) Pub. Date: Oct. 3, 2024

(54) PREPARATION OF SUBSTITUTED AMIDOXIMES

(71) Applicant: BASF SE, Ludwigshafen (DE)

(72) Inventors: Joachim Gebhardt, Ludwigshafen (DE); Guillaume Michel Jacques Garivet, Ludwigshafen (DE); Kailaskumar Borate, Navi Mumbai (IN); Daniel Maximilian Knoll, Ludwigshafen (DE); Roland Goetz, Ludwigshafen (DE)

(21) Appl. No.: 18/576,149

(22) PCT Filed: Jun. 28, 2022
(86) PCT No.: PCT/EP2022/067677

§ 371 Date: Jan. 3, 2024

(30) Foreign Application Priority Data
Jul. 8, 2021 (IN) 202121030667
Sep. 30, 2021 (EP) 21200185.3

(51) Int. Cl. C07D 271/06

(52) U.S. Cl. CPC C07D 271/06

(57) ABSTRACT

The present invention relates to a process for the preparation of substituted amidoximes of formula I, which can be obtained through reaction of nitrile compounds of formula II with free hydroxylamine in the presence of a solvent.

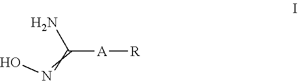

The present invention relates to a process for the preparation of substituted amidoximes of formula I, which can be obtained through reaction of nitrile compounds of formula II with free hydroxylamine in a solvent.

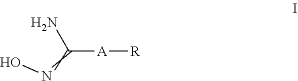

Amidoximes I are versatile synthesis intermediates, which can be converted to substituted 3-aryl-5-trifluoromethyl-1,2,4-oxadiazoles that are known to be useful for controlling phytopathogenic fungi, for example from WO 2015/185485 A1 and WO 2017/211649 A1.

Prior art references often suggest using a large excess of hydroxylamine in the form of one of its acid addition salts, for example hydroxylamine hydrochloride, hydroxylammonium acetate, or hydroxylammonium sulfate for the preparation of amidoximes I from nitriles 1l. These salts are typically employed in syntheses on laboratory scale because they can be handled easily and safely. Hydroxylamine as a free base will readily decompose if not properly stored and handled. Uncontrolled decomposition is rapid and can be violent, generating heat and large quantities of gases that may increase the pressure. This potential increase in pressure can cause the container to rupture with a subsequent release. Dangerous explosive-like decompositions can be caused by overheating, contamination, and/or by concentrating the solution. It was reported that free hydroxylamine may be generated in situ by addition of an auxiliary base to the corresponding acid addition salts. Suitable auxiliary bases can be of organic or inorganic nature, for example, amines, pyridines, carbonates, hydrogencarbonates, hydroxides, or alcoholates. These transformations may also provide satisfactory yields when using approximately equimolar amounts of the hydroxylamine salt based on the amount of nitrile. The following references give representative examples of procedures that form part of the prior art: WO 2015/185485 A1 (working example, step 1) describes the preparation of amidoximes using hydroxylamine hydrochloride and sodium bicarbonate in ethanol at reflux. WO 2017/211649 A1 (working example 1.1) likewise discloses the preparation of amidoximes using hydroxylamine hydrochloride and potassium carbonate in a mixture of ethanol and water at reflux. WO 2010/080357 A1 (working examples, method I, step 2) describes the preparation of amidoximes using an aqueous solution of free hydroxylamine in ethanol as solvent at 80° C.

From an economic point of view it is desirable in industrial large-scale operations dealing with the production of amidoximes I to minimize both, the amount of solvent used in the process and the amount of hydroxylamine. The use of free hydroxylamine is particularly attractive because it i) is more reactive than its salts and provides high yields even if employed in equimolar amounts, ii) is cheaper and more atom-efficient than its salts, iii) does not require the use of a base, iv) leads to significantly smaller waste streams, i.e. without salts, and v) avoids handling of solid material in the process.

When using minimal amounts of a solvent in the process according to the present invention amidoximes of formula I and nitriles of formula II have limited solubility. Thus, at the start of the reaction, the reaction mixture is a suspension containing particulate nitrile II. The inventors found that, in reactions where the mixture obtained in step 1 is a suspension containing particulate nitrile II, the total amount of solid material in the suspension increases over time in step 2, i.e. as the conversion of the nitrile II progresses, which is arguably due to the precipitation of amidoxime 1. This build-up of solid material causes a significant increase of viscosity of the reaction mixture to the point where proper agitation of the reaction mixture eventually gets very hard or is no longer possible resulting in the complete blockage of the reactor. Excessively high levels of viscosity have detrimental effects on the conversion rate and lead to the formation of unwanted side-products.

In view of this, it was an object of the present invention to overcome these disadvantages and to provide an improved and more economical process, which enables the preparation of amidoximes I on an industrial scale in high yield and with low amounts of side-products.

The inventors found that the slow addition of free hydroxylamine to suspensions containing nitrile II surprisingly alleviated the effect of amidoxime precipitation, thus keeping the viscosity of the reaction mixture, technically speaking, at a justifiable level. In this process almost quantitative yields of the desired amidoximes I were obtained.

Hence, the process of the present invention is more cost efficient and eco-friendly than previously reported processes as it employs readily available and cheap reactants in the smallest possible amounts. Furthermore, the present invention demonstrates that fast conversion of the starting material is achieved at comparatively low reaction temperatures.

Accordingly, the present invention relates to a process for preparing amidoximes of formula I,

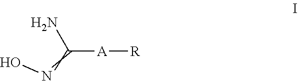

wherein
A is phenyl or a 5- or 6-membered aromatic heterocycle; wherein the ring member atoms of the aromatic heterocycle include besides carbon atoms 1, 2, 3, or 4 heteroatoms selected from N, O, and S as ring member atoms with the provision that the heterocycle cannot contain 2 contiguous atoms selected from O and S; and wherein A is further unsubstituted or further substituted with additional n identical or different radicals R^{A}; wherein
n is 0, 1, 2, 3, or 4;
R^{A1} is independently selected from the group consisting of halogen, cyano, C₁-C₆-alkyl, C₁-C₆-haloalkyl, C₁-C₆-alkoxy, and C₁-C₆-haloalkoxy;
R is methyl, chloromethyl, hydroxymethyl, trichloromethyl, ethyl, iso-propyl, OH, SH, cyano, halogen, CH₂F, CHF₂, 2,2,2-trifluoroethyl, cyclopropyl, —C(═O)H, —C(═NOR²)H, —C(═O)OH, —C(═O)OR¹, —C(═W)N(R¹R²), —CR³R⁴—N(R¹R²), —CR³R⁴—OR¹, —C(═NR¹)R³, —C(═O)R³, —CR³R⁴—C(═O)OH, —CR³R⁴—C(═O)R¹, —CR³R⁴—C(═W)N(R¹R²), —O—CR³R⁴—C(═O)OH, —O—CR³R⁴—C(═O)R¹, —O—CR³R⁴C(═W)N(R¹R²), —CR³R⁴—N(R²)—C(═W)R¹, —CR³R⁴—S(═O)₂R¹, or —CR³R⁴—N(R²)—S(═O)₂R¹; wherein
W is O or S;
R² is hydrogen, C₁-C₆-alkyl, C₂-C₆-alkenyl, C₂-C₆-alkynyl, C₁-C₆-alkoxy, C₃-C₁₁-cycloalkyl, —C(═O)—C₁-C₆-alkyl, —C(═O)—C₃-C₁₁-cycloalkyl, or —C(═O)—O—C₁-C₆-alkyl; and wherein any of the aliphatic or cyclic groups in R² are unsubstituted or substituted with 1, 2, 3, or up to the maximum possible number of identical or different radicals selected from the group consisting of halogen, hydroxy, oxo, cyano, C₁-C₆-alkyl, C₁-C₆-alkoxy, and C₃-C₁₁-cycloalkyl;
R¹ is C₁-C₆-alkyl, C₁-C₆-alkoxy, C₃-C₁₁-cycloalkyl, C₃-C₈-cycloalkenyl, C₂-C₆-alkenyl, C₂-C₆-alkynyl, C₁-C₆-alkoxyimino-C₁-C₄-alkyl, C₂-C₆-alkenyloxyimino-C₁-C₄-alkyl, C₂-C₆-alkynyloxyimino-C₁-C₄-alkyl, C₁-C₆-alkylamino, diC₁-C₆-alkylamino, —C(═O)—C₁-C₆-alkyl, —C(═O)—O—C₁-C₆-alkyl, phenyl-C₁-C₄-alkyl, phenyl-C₁-C₄-alkenyl, phenyl-C₁-C₄-alkynyl, heteroaryl-C₁-C₄-alkyl, phenyl, naphthyl, or a 3- to 10-membered saturated, partially unsaturated or aromatic mono- or bicyclic heterocycle, wherein the ring member atoms of said mono- or bicyclic heterocycle include besides carbon atoms further 1, 2, 3 or 4 heteroatoms selected from N, O and S as ring member atoms with the provision that the heterocycle cannot contain 2 contiguous atoms selected from O and S; and wherein the heteroaryl group in the group heteroaryl-C₁-C₄-alkyl is a 5- or 6-membered aromatic heterocycle, wherein the ring member atoms of the heterocyclic ring include besides carbon atoms 1, 2, 3 or 4 heteroatoms selected from N, O, and S as ring member atoms with the provision that the heterocycle cannot contain 2 contiguous atoms selected from O and S; and wherein any of the above-mentioned aliphatic or cyclic groups are unsubstituted or substituted with 1, 2, 3, or up to the maximum possible number of identical or different groups R^{1a}; or
R¹ and R², together with the nitrogen atom to which they are attached, form a saturated or partially unsaturated mono- or bicyclic 3- to 10-membered heterocycle, wherein the heterocycle includes beside one nitrogen atom and one or more carbon atoms no further heteroatoms or 1, 2 or 3 further heteroatoms independently selected from N, O, and S as ring member atoms with the provision that the heterocycle cannot contain 2 contiguous atoms selected from O and S; and wherein the heterocycle is unsubstituted or substituted with 1, 2, 3, 4, or up to the maximum possible number of identical or different groups R^{1a}; wherein
R^{1a} is halogen, oxo, cyano, NO₂, OH, SH, NH₂, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, —NHSO₂—C₁-C₄-alkyl, —C(═O)—C₁-C₄-alkyl, —C(═O)—O—C₁-C₄-alkyl, C₁-C₆-alkylsulfonyl, hydroxyC₁-C₄-alkyl, —C(═O)—NH₂, —C(═O)—NH(C₁-C₄-alkyl), C₁-C₄-alkylthio-C₁-C₄-alkyl, aminoC₁-C₄-alkyl, C₁-C₄-alkylamino-C₁-C₄-alkyl, diC₁-C₄-alkylamino-C₁-C₄-alkyl, aminocarbonyl-C₁-C₄-alkyl, or C₁-C₄-alkoxy-C₁-C₄-alkyl;
R³, R⁴ independently of each other are selected from the group consisting of hydrogen, halogen, cyano, C₁-C₄-alkyl, C₁-C₄-alkenyl, C₁-C₄-alkynyl, C₁-C₄-haloalkyl and C₁-C₄-alkoxy; or
R³ and R⁴ together with the carbon atom to which they are bound form a cyclopropyl group, by reacting a nitrile of formula II, wherein the variables A and R are as defined above for compounds of formula I,

N≡C-A-R II
with free hydroxylamine in a solvent, the process comprising the following steps:
step 1: charging a reaction vessel with a solvent and nitrile II;
step 2: metering 0.9 to 3.0 equivalents of free hydroxylamine, based on the amount of nitrile II, into the mixture obtained in step 1;
whereas the process is characterized in that the free hydroxylamine in step 2 is metered to nitrile II over a period of equal to or more than 15 minutes.

The general expression “compound I” as used herein is equivalent to the expression “compounds of formula I”. Accordingly, for example, the expression “nitrile of formula II” as used herein is equivalent to the expression “nitrile II”.

The term “free hydroxylamine” as used herein shall mean hydroxylamine (HO—NH₂) in its form as a free base, i.e. in its non-protonated form; whereas “hydroxylammonium salts” are formed with hydroxylamine in the presence of, or upon addition of, an acid, for example, but not limited to, hydrogen chloride, sulfuric acid, or acetic acid.

Since nitrile II may contain small amounts of residual acid from previous synthesis steps, a small amount of hydroxylammonium salts may be present in the process of the present invention. Accordingly, in one aspect of the invention the reaction mixture obtained in step 1 of the process contains less than 0.2 equivalents of hydroxylammonium salts after the addition of the free hydroxylamine to nitrile II, based on the total amount of hydroxylamine in the reaction mixture.

In another aspect of the present invention the reaction mixture obtained in step 1 of the process contains less than 0.1 equivalents of hydroxylammonium salts after the addition of the free hydroxylamine to nitrile II, based on the amount of nitrile II in the reaction mixture.

In a further aspect of the present invention the reaction mixture obtained in step 1 of the process contains less than 0.05 equivalents of hydroxylammonium salts after the addition of the free hydroxylamine to nitrile II, based on the amount of nitrile II in the reaction mixture.

In one embodiment of the present invention 0.9 to 1.5 equivalents of free hydroxylamine are added in step 2 to the reaction mixture obtained in step 1.

In one embodiment of the present invention 1.0 to 1.5 equivalents of free hydroxylamine are added in step 2 to the reaction mixture obtained in step 1.

In another embodiment of the present invention 1.0 to 1.2 equivalents of free hydroxylamine are added in step 2 to the reaction mixture obtained in step 1.

Typically, hydroxylamine is not used in pure form for safety reasons but as an aqueous solution with a concentration of up to 50% (w/w).

In one aspect of the present invention the free hydroxylamine in step 2 is metered to nitrile II over a period of equal to or more than 30 minutes.

In one aspect of the present invention the free hydroxylamine in step 2 is metered to nitrile II over a period of equal to or more than 1 hour.

In one aspect of the present invention the free hydroxylamine in step 2 is metered to nitrile II over a period of equal to or more than 2 hours.

In one aspect of the present invention the free hydroxylamine in step 2 is metered to nitrile II over a period of equal to or more than 3 hours.

In one aspect of the present invention the free hydroxylamine in step 2 is metered to nitrile II over a period of 15 minutes to 48 hours.

In a further aspect of the present invention the free hydroxylamine in step 2 is metered to nitrile II over a period of 15 minutes to 10 hours.

In another aspect of the present invention the free hydroxylamine in step 2 is metered to nitrile II over a period of 15 minutes to 5 hours.

In another aspect of the present invention the free hydroxylamine in step 2 is metered to nitrile II over a period of 30 minutes to 5 hours.

After the addition of the free hydroxylamine the reaction mixture is stirred until the conversion of nitrile II is complete. This process may take between 1 to 24 hours.

In one embodiment the temperature of the reaction mixture in step 2 of the process is between 0° C. and 80° C.

In another embodiment the temperature of the reaction mixture in step 2 of the process is between 20° C. and 60° C.

In a preferred embodiment the temperature of the reaction mixture in step 2 of the process is between 30° C. and 50° C. At temperatures below 50° C. the formation of side-products is sufficiently suppressed.

In a more preferred embodiment the temperature of the reaction mixture in step 2 of the process is between 35° C. and 45° C.

In one aspect of the process the mixture obtained in step 1 and as used in step 2 is a suspension containing particulate nitrile II.

In one embodiment the mixture obtained in step 1 contains equal to or more than 350 g of nitrile II per kilogram of solvent.

In one embodiment the mixture obtained in step 1 contains equal to or more than 450 g of nitrile II per kilogram of solvent.

In another embodiment the mixture obtained in step 1 contains equal to or more than 550 g of nitrile II per kilogram of solvent.

In one aspect of the invention the process is carried out in the absence of an auxiliary base. The term “auxiliary base” as used herein refers to a base, which does not take part in the reaction as a reactant but acts as a scavenger for protons to generate free hydroxylamine from hydroxylammonium salts.

The term “solvent” herein refers to an inert solvent, i.e. a solvent, which is not taking part in the reaction. This means that the auxiliary solvent is not identical with the reactants.

In one aspect of the present invention the process is conducted in a solvent selected from a dipolar organic solvent or water, or mixtures of dipolar organic solvents and water. Suitable dipolar organic solvents are, for example, ethers (diethylether, dibutylether, tert-butylmethylether, ethylene glycol dimethyl ether, ethylene glycol, diethyl ether, diethylene glycol dimethyl ether, 2-methyltetrahydrofuran, tetrahydrofuran, dioxane, diethylene, glycol monomethyl- or monoethyl ether), N-substituted lactams (N-methylpyrrolidone), carboxamides (N,N-dimethylformamide, N,N-dimethylacetamide), acyclic ureas (dimethyl imidazolinum), sulphoxides, sulphones (dimethyl sulfoxide, dimethyl sulfone, tetramethylene sulfoxide, tetramethylene sulfone), and alkyl alcohols (methanol, ethanol, n-propanol, iso-propanol, n-butanol, iso-butanol, sec-butanol, tert-butanol, 1-pentanol, 1-hexanol, 2-ethylhexanol, 3-methyl-1-butanol, cyclohexanol).

In a preferred embodiment the solvent comprises an alkyl alcohol of formula III, or mixtures thereof,

C₁-C₆-alkyl-OH III.

In one embodiment of the present invention, the solvent in the process comprises methanol, ethanol, n-propanol, iso-propanol, n-butanol, iso-butanol, sec-butanol, or tert-butanol.

In a preferred embodiment the solvent comprises methanol or ethanol.

In another particularly preferred embodiment the solvent is methanol or mixtures of methanol with water. In a particularly preferred embodiment the solvent is methanol.

In a particularly preferred embodiment the solvent is ethanol or mixtures of ethanol with water.

In a particularly preferred embodiment the solvent is ethanol.

In a particularly preferred embodiment the process is conducted in a solvent comprising an alkyl alcohol of formula III, more particularly methanol, ethanol, n-propanol, iso-propanol, n-butanol, iso-butanol, sec-butanol, or tert-butanol; wherein 0.9 to 1.5 equivalents of free hydroxylamine is metered in step 2 to the reaction mixture obtained in step 1 over a period of equal to or more than 15 minutes; and wherein the temperature of the reaction mixture in step 2 of the process is between 20° C. to 60° C.

In a particularly preferred embodiment the process is conducted in a solvent comprising an alkyl alcohol of formula III, more particularly methanol, ethanol, n-propanol, iso-propanol, n-butanol, iso-butanol, sec-butanol, or tert-butanol; wherein the mixture obtained in step 1 is a suspension containing particulate nitrile II; wherein 0.9 to 1.5 equivalents of free hydroxylamine is metered in step 2 to the reaction mixture obtained in step 1 over a period of equal to or more than 15 minutes; and wherein the temperature of the reaction mixture in step 2 of the process is between 20° C. to 60° C.

In a particularly preferred embodiment the process is conducted in a solvent comprising an alkyl alcohol of formula III, more particularly methanol, ethanol, n-propanol, iso-propanol, n-butanol, iso-butanol, sec-butanol, or tert-butanol; wherein the mixture obtained in step 1 contains equal to or more than 350 g of nitrile II per kilogram of solvent; wherein 0.9 to 1.5 equivalents of free hydroxylamine is metered in step 2 to the reaction mixture obtained in step 1 over a period of equal to or more than 15 minutes; and wherein the temperature of the reaction mixture in step 2 of the process is between 20° C. to 60° C.

In a further particularly preferred embodiment the process is conducted in a solvent comprising methanol or ethanol; wherein 0.9 to 1.5 equivalents of free hydroxylamine is metered in step 2 to the reaction mixture obtained in step 1 over a period of equal to or more than 15 minutes; and wherein the temperature of the reaction mixture in step 2 of the process is between 20° C. to 60° C.

In a further particularly preferred embodiment the process is conducted in a solvent comprising methanol or ethanol; wherein the mixture obtained in step 1 is a suspension containing particulate nitrile II; wherein 0.9 to 1.5 equivalents of free hydroxylamine is metered in step 2 to the reaction mixture obtained in step 1 over a period of equal to or more than 15 minutes; and wherein the temperature of the reaction mixture in step 2 of the process is between 20° C. to 60° C.

In a further particularly preferred embodiment the process is conducted in a solvent comprising methanol or ethanol; wherein the mixture obtained in step 1 is a suspension containing particulate nitrile II; wherein 0.9 to 1.5 equivalents of free hydroxylamine is metered in step 2 to the reaction mixture obtained in step 1 over a period of equal to or more than 30 minutes; and wherein the temperature of the reaction mixture in step 2 of the process is between 30° C. to 50° C.

In a further particularly preferred embodiment the process is conducted in a solvent comprising methanol or ethanol; wherein the mixture obtained in step 1 contains equal to or more than 350 g of nitrile II per kilogram of solvent; wherein 0.9 to 1.5 equivalents of free hydroxylamine is metered in step 2 to the reaction mixture obtained in step 1 over a period of equal to or more than 15 minutes; and wherein the temperature of the reaction mixture in step 2 of the process is between 20° C. to 60° C.

In a further particularly preferred embodiment the process is conducted in a solvent comprising methanol or ethanol; wherein the mixture obtained in step 1 contains equal to or more than 350 g of nitrile II per kilogram of solvent; wherein 0.9 to 1.5 equivalents of free hydroxylamine is metered in step 2 to the reaction mixture obtained in step 1 over a period of equal to or more than 30 minutes; and wherein the temperature of the reaction mixture in step 2 of the process is between 30° C. to 50° C.

In still another preferred embodiment the process is conducted in a solvent comprising methanol or ethanol; wherein the mixture obtained in step 1 contains equal to or more than 450 g of nitrile II per kilogram of solvent; wherein 1.0 to 1.5 equivalents of free hydroxylamine is metered in step 2 to the reaction mixture obtained in step 1 over a period of equal to or more than 30 minutes; and wherein the temperature of the reaction mixture in step 2 of the process is between 30° C. to 50° C.

The process of the present invention is typically carried out at atmospheric pressure. After the addition of the free hydroxylamine is complete, water is slowly added to the reaction mixture in order to reduce product solubility for work up. The reaction mixture is then cooled to 0-5° C. before filtration in order to avoid product losses in the mother liquor.

In one aspect of the present invention the variable A is phenyl in compounds of formula I and II.

In one embodiment of the present invention radical R^{A} in compounds of formula I and ∥ is halogen, C₁-C₆-alkyl, C₁-C₆-haloalkyl, C₁-C₆-alkoxy, or C₁-C₆-haloalkoxy; particularly fluorine.

In one aspect n is 1 and R^{A} is fluorine in compounds of formula I and |1.

In a preferred embodiment the variable n is 0 in compounds of formula I and II.

In one aspect the present invention relates to a process as defined above, wherein the nitrile is of formula II.b,

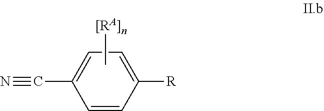

wherein n is 0 or 1; and wherein the meaning of R is as defined or preferably defined herein for compounds of formula I; and wherein R^{A} is selected from the group consisting of halogen, C₁-C₆-alkyl, C₁-C₆-haloalkyl, C₁-C₆-alkoxy, and C₁-C₆-haloalkoxy; to obtain amidoximes of formula I.b,

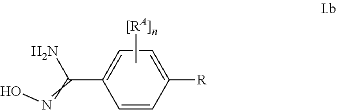

wherein the variables n, R^{A}, and R have the meaning as defined for compounds II.b.

In another embodiment n is 1 and R^{A} is fluorine in compounds of formula I.b and II.b.

In a preferred embodiment n is 0 in compounds of formula I.b and II.b.

In one embodiment the variables in compounds of formula I, II, Ib, and II.b have the following meaning:
R^{A1} is fluorine
n is 0 or 1;
R is methyl, chloromethyl, hydroxymethyl, trichloromethyl, —C(═O)H, —C(═NOR²)H, —C(═O)OH, OH, SH, cyano, halogen, —C(═O)NR¹R², —CH₂—N(R²)—C(═O)R¹, —CH₂—N(R²)—S(═O)₂R¹,

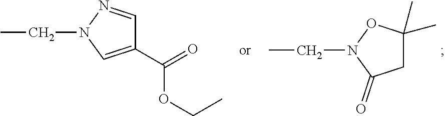

R¹ is methyl, ethyl, n-propyl, iso-propyl, n-butyl, sec-butyl, iso-butyl, cyclopropyl, 2-methoxyiminoethyl, bicyclo[1.1.1]pentan-1-yl, or phenyl; and wherein the phenyl group is unsubstituted or substituted with 1, 2, 3 or up to the maximum possible number of identical or different radicals selected from the group consisting of fluorine, chlorine, cyano, methyl, ethyl, methoxy, trifluoromethyl, trifluoromethoxy, difluoromethyl, difluoromethoxy, and cyclopropyl;
R² is hydrogen, methyl, ethyl, methoxy, ethoxy, or cyclopropyl.

In a further embodiment the variables in compounds of formula I, II, I.b, and II.b have the following meaning:
R^{A1} is fluorine
n is 0 or 1;
R is methyl, —C(═O)OH, —C(═O)NR¹R², —CH₂—N(R²)—C(═O)R¹, —CH₂—N(R²)—S(═O)₂R¹,

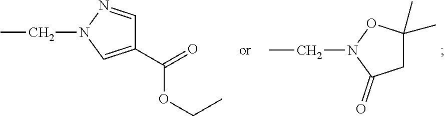

R¹ is C₁-C₆-alkly, phenyl, or cyclopropyl, wherein the phenyl ring is unsubstituted or substituted with 1, 2, 3, or 4 identical or different groups selected from halogen;
R² is hydrogen, methyl, ethyl, methoxy, ethoxy, or cyclopropyl.

In yet another embodiment the variables in compounds of formula I, II, I.b, and II.b have the following meaning:
R^{A1} is fluorine
n is 0 or 1;
R is —CH₂—N(R²)—C(═O)R¹, —CH₂—N(R²)—S(═O)₂R¹,

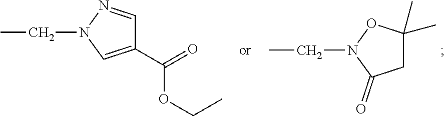

R¹ is C₁-C₆-alkly, or cyclopropyl;
R² is hydrogen, methyl, methoxy, ethoxy, or cyclopropyl.

In another embodiment the variables in compounds of formula I, II, I.b, and II.b have the following meaning:
R^{A1} is fluorine
n is 0 or 1;
R is methyl, —C(═O)OH, or —C(═O)NR¹R²;
R¹ is methyl or phenyl, wherein the phenyl ring is unsubstituted or substituted with 1, 2, 3, or 4 identical or different groups selected from halogen;
R² is hydrogen, methyl, ethyl, methoxy, or ethoxy.

In still another embodiment the variables in compounds of formula I, II, I.b, and II.b have the following meaning:
n is 0;
R is —C(═O)NR¹R²;
R¹ is methyl, 2-methoxyiminoethyl, bicyclo[1.1.1]pentan-1-yl, 2-fluoro-phenyl, 4-fluoro-phenyl, or 2,4-difluorophenyl; in particular methyl or 2-fluoro-phenyl;
R² is hydrogen.

In a particularly advantageous approach a compound of formula I.b, wherein n is 0, may be reacted with an activated form of trifluoroacetic acid (for example using trifluoro acetic acid anhydride, trifluoroacetic acid halides, or using methyl or ethyl trifluoroacetate, for reference see WO 2015/185485 A1, WO 2019/02045 A1, PCT/EP2021/052256 and WO 2020/212513 A1) to obtain the compound of formula IV,

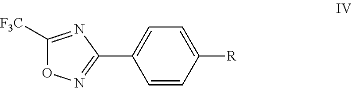

wherein the variable R has the meaning as defined or preferably defined herein for compounds I.b. This two-step transformation is particularly preferred in regard to compounds IV and I.b, wherein R is —C(═O)N(R¹R²), R¹ is hydrogen, and R² is 2-fluorophenyl.

In a further embodiment of the invention a compound of formula I.b, wherein R is methyl, is converted into valuable chemical products or intermediates. Accordingly, in one embodiment, compounds of formula I.b, wherein n is 0 and R is methyl, can be further chlorinated to obtain a compound of formula IV.a.

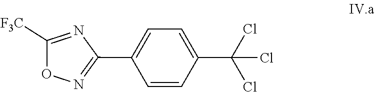 [SMILES: FC(F)(F)c1nc(-c2ccc(C(Cl)(Cl)Cl)cc2)no1]

The chlorination of the methyl group R of compounds of formula I.b can be achieved as described in WO 2019/020451 A1 and the references cited therein.

In a further embodiment the compound of formula IV.a is hydrolyzed to obtain a compound of formula V.

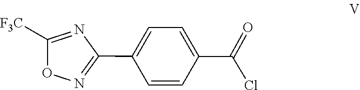 [SMILES: O=C(Cl)c1ccc(-c2noc(C(F)(F)F)n2)cc1]

In one embodiment this transformation is carried out in the presence of catalytic amounts of a Lewis acid and water to obtain a compound of formula V, as described in WO 2019/020451 A1 and the references cited therein. Preferably, the Lewis acid is a metal salt, for example aluminum(III) chloride or iron(III) chloride, particularly iron(III) chloride.

In another embodiment the compound of formula I.b, wherein n is 0 and R is —C(═O)OH, is chlorinated to obtain a compound of formula V.

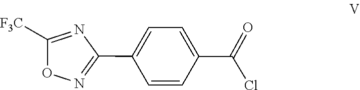 [SMILES: O=C(Cl)c1ccc(-c2noc(C(F)(F)F)n2)cc1]

These transformations are described in WO 2019/020451 A1 and WO 2017/211649 A1 and the references cited therein.

In one embodiment, the compound of formula V is reacted with an amine of formula VI,

R¹—NH—R² VI
wherein
R¹ is C₁-C₆-alkyl, C₁-C₆-alkoxy, C₃-C₁₁-cycloalkyl, C₃-C₈-cycloalkenyl, C₂-C₆-alkenyl, C₂-C₆-alkynyl, C₁-C₆-alkoxyimino-C₁-C₄-alkyl, C₂-C₆-alkenyloxyimino-C₁-C₄-alkyl, C₂-C₆-alkynyloxyimino-C₁-C₄-alkyl, C₁-C₆-alkylamino, diC₁-C₆-alkylamino, —C(═O)—C₁-C₆-alkyl, —C(═O)—O—C₁-C₆-alkyl, phenyl-C₁-C₄-alkyl, phenyl-C₁-C₄-alkenyl, phenyl-C₁-C₄-alkynyl, heteroaryl-C₁-C₄-alkyl, phenyl, naphthyl, or a 3- to 10-membered saturated, partially unsaturated or aromatic mono- or bicyclic heterocycle, wherein the ring member atoms of said mono- or bicyclic heterocycle include besides carbon atoms further 1, 2, 3 or 4 heteroatoms selected from N, O and S as ring member atoms with the provision that the heterocycle cannot contain 2 contiguous atoms selected from O and S; and wherein the heteroaryl group in the group heteroaryl-C₁-C₄-alkyl is a 5- or 6-membered aromatic heterocycle, wherein the ring member atoms of the heterocyclic ring include besides carbon atoms 1, 2, 3 or 4 heteroatoms selected from N, O, and S as ring member atoms with the provision that the heterocycle cannot contain 2 contiguous atoms selected from 0 and S; and wherein any of the above-mentioned aliphatic or cyclic groups are unsubstituted or substituted with 1, 2, 3, or up to the maximum possible number of identical or different groups R^{1a}; or
R¹ and R², together with the nitrogen atom to which they are attached, form a saturated or partially unsaturated mono- or bicyclic 3- to 10-membered heterocycle, wherein the heterocycle includes beside one nitrogen atom and one or more carbon atoms no further heteroatoms or 1, 2 or 3 further heteroatoms independently selected from N, O, and S as ring member atoms with the provision that the heterocycle cannot contain 2 contiguous atoms selected from O and S; and wherein the heterocycle is unsubstituted or substituted with 1, 2, 3, 4, or up to the maximum possible number of identical or different groups R^{1a}; wherein
R^{1a} is halogen, oxo, cyano, NO₂, OH, SH, NH₂, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, —NHSO₂—C₁-C₄-alkyl, (C═O)—C₁-C₆-alkyl, C(═O)—O—C₁-C₆-alkyl, C₁-C₆-alkylsulfonyl, hydroxyC₁-C₄-alkyl, C(═O)—NH₂, C(═O)—NH(C₁-C₄-alkyl), C₁-C₄-alkylthio-C₁-C₄-alkyl, aminoC₁-C₄-alkyl, C₁-C₄-alkylamino-C₁-C₄-alkyl, diC₁-C₄-alkylamino-C₁-C₄-alkyl, aminocarbonyl-C₁-C₄-alkyl, or C₁-C₄-alkoxy-C₁-C₄-alkyl;
R² is hydrogen, C₁-C₆-alkyl, C₂-C₆-alkenyl, C₂-C₆-alkynyl, C₁-C₆-alkoxy, C₃-C₁₁-cycloalkyl, —C(═O)H, —C(═O)—C₁-C₆-alkyl, —C(═O)—C₃-C₁₁-cycloalkyl, or —C(═O)—O—C₁-C₆-alkyl; and wherein any of the aliphatic or cyclic groups in R² are unsubstituted or substituted with 1, 2, 3, or up to the maximum possible number of identical or different radicals selected from the group consisting of halogen, hydroxy, oxo, cyano, C₁-C₆-alkyl, C₁-C₆-alkoxy, and C₃-C₁₁-cycloalkyl;
to obtain a compound of formula VII.

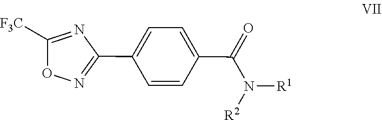

These transformations are also described in WO 2019/020451 A1 and WO 2017/211652 A1 and the references cited therein.

In another embodiment, the compound of formula VII is used to obtain a compound of formula VIII,

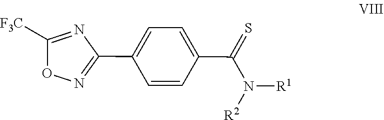

as described in WO 2019/020451 A1 and WO 2017/211649 A1 and the references cited therein.

In a preferred embodiment the variables R¹ and R² in compounds of formula I, Ib, II.b, IV, VII and VIII have the following meaning:
R¹ is methyl, ethyl, n-propyl, iso-propyl, n-butyl, sec-butyl, iso-butyl, cyclopropyl, 2-methoxyiminoethyl, bicyclo[1.1.1]pentan-1-yl, or phenyl; and wherein the phenyl group is unsubstituted or substituted with 1, 2, 3 or up to the maximum possible number of identical or different radicals selected from the group consisting of fluorine, chlorine, cyano, OH, NH₂, methyl, ethyl, methoxy, trifluoromethyl, trifluoromethoxy, difluoromethyl, difluoromethoxy, and cyclopropyl; and
R² is hydrogen, methyl, or ethyl.

In another preferred embodiment the variables R¹ and R² in compounds of formula I, Ib, II.b, IV, VII and VIII have the following meaning:
R¹ is methyl, 2-methoxyiminoethyl, bicyclo[1.1.1]pentan-1-yl, 2-fluoro-phenyl, 4-fluoro-phenyl, or 2,4-difluorophenyl; in particular methyl or 2-fluoro-phenyl; and
R² is hydrogen.

In the definitions of the variables given above, collective terms are used which are generally representative for the substituents in question.

The term “Cₙ-Cₘ” indicates the number of carbon atoms possible in each case in the substituent or substituent moiety in question.

The term “halogen” refers to fluorine, chlorine, bromine and iodine.

The term “oxo” refers to an oxygen atom ═O, which is bound to a carbon atom or sulfur atom, thus forming, for example, a ketonyl —C(═O)— or sulfinyl —S(═O)— group.

The term “C₁-C₆-alkyl” refers to a straight-chained or branched saturated hydrocarbon group having 1 to 6 carbon atoms, for example methyl, ethyl, propyl, 1-methylethyl, butyl, 1-methylpropyl, 2-methylpropyl, and 1,1-dimethylethyl.

The term “C₂-C₆-alkenyl” refers to a straight-chain or branched unsaturated hydrocarbon radical having 2 to 6 carbon atoms and a double bond in any position, such as ethenyl, 1-propenyl, 2-propenyl (allyl), 1-methylethenyl, 1-butenyl, 2-butenyl, 3-butenyl, 1-methyl-1-propenyl, 2-methyl-1-propenyl, 1-methyl-2-propenyl, 2-methyl-2-propenyl.

The term “C₂-C₆-alkynyl” refers to a straight-chain or branched unsaturated hydrocarbon radical having 2 to 6 carbon atoms and containing at least one triple bond, such as ethynyl, 1-propynyl, 2-propynyl (propargyl), 1-butynyl, 2-butynyl, 3-butynyl, 1-methyl-2-propynyl.

The term “C₁-C₆-haloalkyl” refers to a straight-chained or branched alkyl group having 1 to 6 carbon atoms (as defined above), wherein some or all of the hydrogen atoms in these groups may be replaced by halogen atoms as mentioned above, for example chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl and pentafluoroethyl, 2-fluoropropyl, 3-fluoropropyl, 2,2-difluoropropyl, 2,3-difluoropropyl, 2-chloropropyl, 3-chloropropyl, 2,3-dichloropropyl, 2-bromopropyl, 3-bromopropyl, 3,3,3-trifluoropropyl, 3,3,3-trichloropropyl, CH₂—C₂F₅, CF₂—C₂F₅, CF(CF₃)₂, 1-(fluoromethyl)-2-fluoroethyl, 1-(chloromethyl)-2-chloroethyl, 1-(bromomethyl)-2-bromoethyl, 4-fluorobutyl, 4-chlorobutyl, 4-bromobutyl or nonafluorobutyl.

The term “C₁-C₆-alkoxy” refers to a straight-chain or branched alkyl group having 1 to 6 carbon atoms (as defined above) which is bonded via an oxygen, at any position in the alkyl group, for example methoxy, ethoxy, n-propoxy, 1-methylethoxy, butoxy, 1-methylpropoxy, 2-methylpropoxy or 1,1-dimethylethoxy.

The term “C₁-C₆-haloalkoxy” refers to a C₁-C₆-alkoxy group as defined above, wherein some or all of the hydrogen atoms may be replaced by halogen atoms as mentioned above, for example, OCH₂F, OCHF₂, OCF₃, OCH₂Cl, OCHCl₂, OCCl₃, chlorofluoromethoxy, dichlorofluoromethoxy, chlorodifluoromethoxy, 2-fluoroethoxy, 2-chloroethoxy, 2-bromoethoxy, 2-iodoethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy, OC₂F₅, 2-fluoropropoxy, 3-fluoropropoxy, 2,2-difluoropropoxy, 2,3-difluoropropoxy, 2-chloropropoxy, 3-chloropropoxy, 2,3-dichloropropoxy, 2-bromopropoxy, 3-bromopropoxy, 3,3,3-trifluoropropoxy, 3,3,3-trichloropropoxy, OCH₂—C₂F₅, OCF₂—C₂F₅, 1-(CH₂F)-2-fluoroethoxy, 1-(CH₂Cl)-2-chloroethoxy, 1-(CH₂Br)-2-bromoethoxy, 4-fluorobutoxy, 4-chlorobutoxy, 4-bromobutoxy or nonafluorobutoxy.

The terms “phenyl-C₁-C₄-alkyl or heteroaryl-C₁-C₄-alkyl” refer to alkyl having 1 to 4 carbon atoms (as defined above), wherein one hydrogen atom of the alkyl radical is replaced by a phenyl or hetereoaryl radical respectively.

The term “C₁-C₄-alkoxy-C₁-C₄-alkyl” refers to alkyl having 1 to 4 carbon atoms (as defined above), wherein one hydrogen atom of the alkyl radical is replaced by a C₁-C₄-alkoxy group (as defined above). Likewise, the term “C₁-C₄-alkylthio-C₁-C₄-alkyl” refers to alkyl having 1 to 4 carbon atoms (as defined above), wherein one hydrogen atom of the alkyl radical is replaced by a C₁-C₄-alkylthio group.

The term “C₁-C₆-alkylthio” as used herein refers to straight-chain or branched alkyl groups having 1 to 6 carbon atoms (as defined above) bonded via a sulfur atom. Accordingly, the term “C₁-C₆-haloalkylthio” as used herein refers to straight-chain or branched haloalkyl group having 1 to 6 carbon atoms (as defined above) bonded through a sulfur atom, at any position in the haloalkyl group.

The term “C₁-C₄-alkoxyimino” refers to a divalent imino radical (C₁-C₄-alkyl-O—N═) carrying one C₁-C₄-alkoxy group as substituent, e.g. methylimino, ethylimino, propylimino, 1-methylethyl-imino, butylimino, 1-methylpropylimino, 2-methylpropylimino, 1,1-dimethylethylimino and the like.

The term “C₁-C₆-alkoxyimino-C₁-C₄-alkyl” refers to alkyl having 1 to 4 carbon atoms, wherein two hydrogen atoms of one carbon atom of the alkyl radical are replaced by a divalent C₁-C₆-alkoxyimino radical (C₁-C₆-alkyl-O—N═) as defined above.

The term “C₂-C₆-alkenyloxyimino-C₁-C₄-alkyl” refers to alkyl having 1 to 4 carbon atoms, wherein two hydrogen atoms of one carbon atom of the alkyl radical are replaced by a divalent C₂-C₆-alkenyloxyimino radical (C₂-C₆-alkenyl-O—N═).

The term “C₂-C₆-alkynyloxyimino-C₁-C₄-alkyl” refers to alkyl having 1 to 4 carbon atoms, wherein two hydrogen atoms of one carbon atom of the alkyl radical are replaced by a divalent C₂-C₆-alkynyloxyimino radical (C₂-C₆-alkynyl-O—N═).

The term “hydroxyC₁-C₄-alkyl” refers to alkyl having 1 to 4 carbon atoms, wherein one hydrogen atom of the alkyl radical is replaced by a OH group.

The term “aminoC₁-C₄-alkyl” refers to alkyl having 1 to 4 carbon atoms, wherein one hydrogen atom of the alkyl radical is replaced by a NH₂ group.

The term “C₁-C₆-alkylamino” refers to an amino group, which is substituted with one residue independently selected from the group that is defined by the term C₁-C₆-alkyl. Likewise, the term “diC₁-C₆-alkylamino” refers to an amino group, which is substituted with two residues independently selected from the group that is defined by the term C₁-C₆-alkyl.

The term “C₁-C₄-alkylamino-C₁-C₄-alkyl” refers to refers to alkyl having 1 to 4 carbon atoms (as defined above), wherein one hydrogen atom of the alkyl radical is replaced by a C₁-C₄-alkyl-NH-group which is bound through the nitrogen. Likewise, the term “diC₁-C₄-alkylamino-C₁-C₄-alkyl” refers to refers to alkyl having 1 to 4 carbon atoms (as defined above), wherein one hydrogen atom of the alkyl radical is replaced by a (C₁-C₄-alkyl)₂N— group which is bound through the nitrogen.

The term “aminocarbonyl-C₁-C₄-alkyl” refers to alkyl having 1 to 4 carbon atoms, wherein one hydrogen atom of the alkyl radical is replaced by a —(C═O)—NH₂ group.

The term “C₃-C₁₁-cycloalkyl” refers to a monocyclic, bicyclic or tricyclic saturated univalent hydrocarbon radical having 3 to 11 carbon ring members that is connected through one of the ring carbon atoms by substitution of one hydrogen atom, such as cyclopropyl (C₃H₅), cyclobutyl, cyclopentyl, cyclohexyl, cycloheptyl, cyclooctyl, bicyclo[1.1.0]butyl, bicyclo[2.1.0]pentyl, bicyclo[1.1.1]pentyl, bicyclo[3.1.0]hexyl, bicyclo[2.1.1]hexyl, norcaranyl (bicyclo[4.1.0]heptyl) and norbornyl (bicyclo[2.2.1]heptyl).

The terms “—C(═O)—C₁-C₆-alkyl”, “—C(═O)—O—C₁-C₆-alkyl” and “—C(═O)—C₃-C₁₁-cycloalkyl” refer to aliphatic radicals which are attached through the carbon atom of the —C(═O)— group.

The term “aliphatic” refers to compounds or radicals composed of carbon and hydrogen and which are non-aromatic compounds. An “alicyclic” compound or radical is an organic compound that is both aliphatic and cyclic. They contain one or more all-carbon rings which may be either saturated or unsaturated, but do not have aromatic character.

The terms “cyclic moiety” or “cyclic group” refer to a radical which is an alicyclic ring or an aromatic ring, such as, for example, phenyl or heteroaryl.

The term “and wherein any of the aliphatic or cyclic groups are unsubstituted or substituted with . . . ” refers to aliphatic groups, cyclic groups and groups, which contain an aliphatic and a cyclic moiety in one group, such as in, for example, C₃-C₈-cycloalkyl-C₁-C₄-alkyl; therefore a group which contains an aliphatic and a cyclic moiety both of these moieties may be substituted or unsubstituted independently of each other.

The term “phenyl” refers to an aromatic ring systems including six carbon atoms (commonly referred to as benzene ring.

The term “heteroaryl” refers to aromatic monocyclic or polycyclic ring systems including besides carbon atoms, 1, 2, 3 or 4 heteroatoms independently selected from the group consisting of N, O and S.

The term “saturated 3- to 7-membered carbocycle” is to be understood as meaning monocyclic saturated carbocycles having 3, 4 or 5 carbon ring members. Examples include cyclopropyl, cyclopentyl, cyclohexyl, cycloheptyl, and the like.

The term “3- to 10-membered saturated, partially unsaturated or aromatic mono- or bicyclic heterocycle, wherein the ring member atoms of said mono- or bicyclic heterocycle include besides carbon atoms further 1, 2, 3 or 4 heteroatoms selected from N, O and S as ring member atoms”, is to be understood as meaning both, aromatic mono- and bicyclic heteroaromatic ring systems, and also saturated and partially unsaturated heterocycles, for example:
a 3- or 4-membered saturated heterocycle which contains 1 or 2 heteroatoms from the group consisting of N, O and S as ring members such as oxirane, aziridine, thiirane, oxetane, azetidine, thiethane, [1,2]dioxetane, [1,2]dithietane, [1,2]diazetidine;
and a 5- or 6-membered saturated or partially unsaturated heterocycle which contains 1, 2 or 3 heteroatoms from the group consisting of N, O and S as ring members such as 2-tetrahydrofuranyl, 3-tetrahydrofuranyl, 2-tetrahydrothienyl, 3-tetrahydrothienyl, 2-pyrrolidinyl, 3-pyrrolidinyl, 3-isoxazolidinyl, 4-isoxazolidinyl, 5-isoxazolidinyl, 3-isothiazolidinyl, 4-isothiazolidinyl, 5-isothiazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 2-imidazolidinyl, 4-imidazolidinyl, 1,2,4-oxadiazolidin-3-yl, 1,2,4-oxadiazolidin-5-yl, 1,2,4-thiadiazolidin-3-yl, 1,2,4-thiadiazolidin-5-yl, 1,2,4-triazolidin-3-yl, 1,3,4-oxadiazolidin-2-yl, 1,3,4-thiadiazolidin-2-yl, 1,3,4-triazolidin-2-yl, 2,3-dihydrofur-2-yl, 2,3-dihydrofur-3-yl, 2,4-dihydrofur-2-yl, 2,4-dihydrofur-3-yl, 2,3-dihydrothien-2-yl, 2,3-dihydrothien-3-yl, 2,4-dihydrothien-2-yl, 2,4-dihydrothien-3-yl, 2-pyrrolin-2-yl, 2-pyrrolin-3-yl, 3-pyrrolin-2-yl, 3-pyrrolin-3-yl, 2-isoxazolin-3-yl, 3-isoxazolin-3-yl, 4-isoxazolin-3-yl, 2-isoxazolin-4-yl, 3-isoxazolin-4-yl, 4-isoxazolin-4-yl, 2-isoxazolin-5-yl, 3-isoxazolin-5-yl, 4-isoxazolin-5-yl, 2-isothiazolin-3-yl, 3-isothiazolin-3-yl, 4-isothiazolin-3-yl, 2-isothiazolin-4-yl, 3-isothiazolin-4-yl, 4-isothiazolin-4-yl, 2-isothiazolin-5-yl, 3-isothiazolin-5-yl, 4-isothiazolin-5-yl, 2,3-dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 3,4-dihydrooxazol-2-yl, 3,4-dihydrooxazol-3-yl, 3,4-dihydrooxazol-4-yl, 2-piperidinyl, 3-piperidinyl, 4-piperidinyl, 1,3-dioxan-5-yl, 2-tetrahydropyranyl, 4-tetrahydropyranyl, 2-tetrahydrothienyl, 3-hexahydropyridazinyl, 4-hexahydropyridazinyl, 2-hexahydropyrimidinyl, 4-hexahydropyrimidinyl, 5-hexahydropyrimidinyl, 2-piperazinyl, 1,3,5-hexahydrotriazin-2-yl and 1,2,4-hexahydrotriazin-3-yl and also the corresponding -ylidene radicals; and a 7-membered saturated or partially unsaturated heterocycle such as tetra- and hexahydroazepinyl, such as 2,3,4,5-tetrahydro[1H]azepin-1-,-2-,-3-,-4-,-5-,-6- or -7-yl, 3,4,5,6-tetrahydro[2H]azepin-2-,-3-,-4-,-5-,-6- or -7-yl, 2,3,4,7-tetrahydro[1H]azepin-1-,-2-,-3-,-4-,-5-,-6- or -7-yl, 2,3,6,7-tetrahydro[1H]azepin-1-,-2-,-3-,-4-,-5-,-6- or -7-yl, hexahydroazepin-1-,-2-,-3- or -4-yl, tetra- and hexahydrooxepinyl such as 2,3,4,5-tetrahydro[1H]oxepin-2-,-3-,-4-,-5-,-6- or -7-yl, 2,3,4,7-tetrahydro[1H]oxepin-2-,-3-,-4-,-5-,-6- or -7-yl, 2,3,6,7-tetrahydro[1H]oxepin-2-, -3-,-4-,-5-,-6- or -7-yl, hexahydroazepin-1-,-2-,-3- or -4-yl, tetra- and hexahydro-1,3-diazepinyl, tetra- and hexahydro-1,4-diazepinyl, tetra- and hexahydro-1,3-oxazepinyl, tetra- and hexahydro-1,4-oxazepinyl, tetra- and hexahydro-1,3-dioxepinyl, tetra- and hexahydro-1,4-dioxepinyl and the corresponding -ylidene radicals.

The term “5- or 6-membered heteroaryl” or the term “5- or 6-membered aromatic heterocycle” refer to aromatic ring systems including besides carbon atoms, 1, 2, 3 or 4 heteroatoms independently selected from the group consisting of N, O and S, for example, a 5-membered heteroaryl such as pyrrol-1-yl, pyrrol-2-yl, pyrrol-3-yl, thien-2-yl, thien-3-yl, furan-2-yl, furan-3-yl, pyrazol-1-yl, pyrazol-3-yl, pyrazol-4-yl, pyrazol-5-yl, imidazol-1-yl, imidazol-2-yl, imidazol-4-yl, imidazol-5-yl, oxazol-2-yl, oxazol-4-yl, oxazol-5-yl, isoxazol-3-yl, isoxazol-4-yl, isoxazol-5-yl, thiazol-2-yl, thiazol-4-yl, thiazol-5-yl, isothiazol-3-yl, isothiazol-4-yl, isothiazol-5-yl, 1,2,4-triazolyl-1-yl, 1,2,4-triazol-3-yl 1,2,4-triazol-5-yl, 1,2,4-oxadiazol-3-yl, 1,2,4-oxadiazol-5-yl and 1,2,4-thiadiazol-3-yl, 1,2,4-thiadiazol-5-yl; or
a 6-membered heteroaryl, such as pyridin-2-yl, pyridin-3-yl, pyridin-4-yl, pyridazin-3-yl, pyridazin-4-yl, pyrimidin-2-yl, pyrimidin-4-yl, pyrimidin-5-yl, pyrazin-2-yl and 1,3,5-triazin-2-yl and 1,2,4-triazin-3-yl.

### WORKING EXAMPLES

The present invention is further illustrated by means of the following working examples.

Analytical method 1: HPLC Agilent 1100 Series; column: Agilent Zorbax Phenyl-Hexyl 1.8 μm 50*4.6 mm, Column Flow: 1 mL/min, time: 25 min, pressure: 20000 kPa; temperature: 20° C.; wavelength 200 nm; injector volume: 2 uL; retention time of the respective products is based on reference material.

Eluent: A: Water with 0.1 vol % H₃PO₄; B: Acetonitrile

| Time (min) | % B | Rate (mL/min) |
|---|---|---|
| 0.0 | 14 | 1.0 |
| 16.0 | 86 | 1.0 |
| 20.0 | 86 | 1.0 |
| 20.1 | 14 | 1.0 |

### Example 1) Preparation of N-(2-fluorophenyl)-4-(N′-hydroxycarbamimidoyl)benzamide

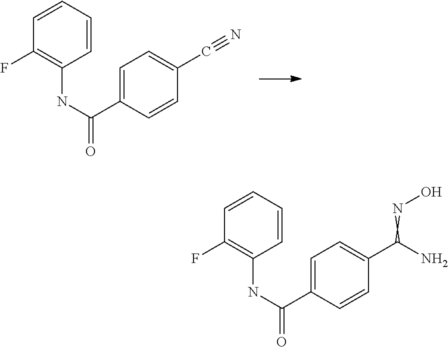

A reaction vessel was charged at room temperature with 816 g methanol (15 mol) and 408.3 g 4-cyano-N-(2-fluorophenyl)benzamide (1.7 mol). The reaction mixture was heated to 40° C. and then 129.2 g hydroxylamine (1.955 mol, 50% in water) was added within 180 minutes at 40° C. The resulting suspension was well agitable without further methanol addition. The mixture was stirred for additional 12 hours at 40° C. Then, 816 g water was added at 40° C. within 1 hour. The resulting mixture was cooled to 5° C. within 3 hours. The precipitated solid was filtered off, washed with 816 g water and dried under reduced pressure (50 mbar) at 70° C. 467.2 g of product were obtained. (purity: 98.1% (w/w, determined by quantitative HPLC analytics), yield 98.7%).

### Example 2) Preparation of N-(2-fluorophenyl)-4-(N′-hydroxycarbamimidoyl)benzamide

A reaction vessel was charged at room temperature with 118.7 g methanol (3.7 mol), 60.0 g 4-cyano-N-(2-fluorophenyl)benzamide (0.247 mol) and 0.3 g sulfuric acid (98%, 0.003 mol). The reaction mixture was heated to 40° C. and then 18.8 g hydroxylamine (0.284 mol, 50% in water) was continuously dosed over a time period of 4 hours at 40° C. The mixture was well agitable over the whole time (agitator with 600 rpm) and poststirred over 9 hours at 40° C. Then, 170 g water was added at 40° C. The resulting mixture was cooled to room temperature. The precipitated solid was filtered off, washed with 100 g water and dried under reduced pressure (50 mbar) at 80° C. 64.5 g of product were obtained. (purity: 96.9% (w/w, determined by quantitative HPLC analytics), yield 92.5%).

### Example 3) Preparation of N-(2-fluorophenyl)-4-(N′-hydroxycarbamimidoyl)benzamide

A reaction vessel was charged at room temperature with 118.7 g methanol (3.7 mol), 60.0 g 4-cyano-N-(2-fluorophenyl)benzamide (0.247 mol) and 0.3 g sulfuric acid (98%, 0.003 mol). The reaction mixture was heated to 40° C. and then 18.8 g hydroxylamine (0.284 mol, 50% in water) was added within less than 1 minute at 40° C. The suspension was getting very viscous after 1 h and agitation was not feasible anymore (agitator with 600 rpm did not mix the visible vessel content anymore—such process cannot be scaled up for production). The mixture was nevertheless “poststirred” over 12 hours at 40° C. Then, 170 g water was added at 40° C. The resulting mixture was cooled to room temperature. The precipitated solid was filtered off, washed with 100 g water and dried under reduced pressure (50 mbar) at 80° C. 63.3 g of product were obtained. (purity: 96.7% (w/w, determined by quantitative HPLC analytics), yield 90.6%).

### Example 4) Preparation of N-(2-fluorophenyl)-4-[5-(trifluoromethyl)-1,2,4-oxadiazol-3-yl]benzamide

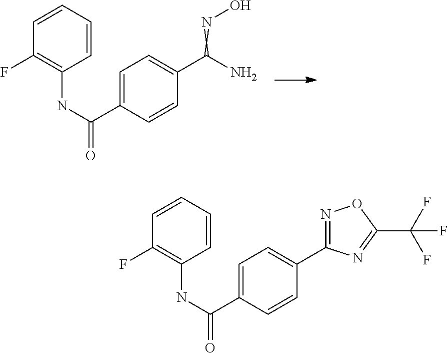

A flask was charged with 500 mg (1.83 mmol) of N-(2-fluorophenyl)-4-[(Z)—N′-hydroxycarbamimidoyl]benzamide and 5 mL of N,N-dimethylformamide at room temperature. To this reaction mass 416 mg (2.93 mmol) of ethyl trifluoroacetate was added at room temperature followed by dropwise addition of 527 mg sodium methanolate (2.93 mmol, 30% w/w in methanol). Slight exotherm was observed during this addition and the reaction mass turned reddish brown. The reaction mass was stirred at room temperature for 1 further hour. HPLC analysis confirmed complete conversion. Then, water was added to the reaction mass, which caused the product to precipitate. The product was filtered and the filter cake was washed with water to remove N,N-dimethylformamide followed by drying to yield 0.53 g (84.6%, purity determined by quantitative HPLC analytics: 97.2%) of the title compound.

1. A process for preparing an amidoxime compound of formula I, 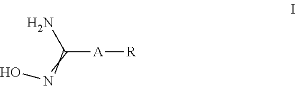
wherein
A is phenyl or a 5- or 6-membered aromatic heterocycle; wherein ring member atoms of the aromatic heterocycle include besides carbon atoms 1, 2, 3, or 4 heteroatoms selected from N, O, and S as ring member atoms with the provision that the heterocycle cannot contain 2 contiguous atoms selected from O and S; and wherein A is unsubstituted or substituted with additional n identical or different radicals R^{A}; wherein
n is 0, 1, 2, 3, or 4;
R^{A1} is independently selected from the group consisting of halogen, cyano, C₁-C₆-alkyl, C₁-C₆-haloalkyl, C₁-C₆-alkoxy, and C₁-C₆-haloalkoxy;
R is methyl, chloromethyl, hydroxymethyl, trichloromethyl, ethyl, iso-propyl, OH, SH, cyano, halogen, CH₂F, CHF₂, 2,2,2-trifluoroethyl, cyclopropyl, —C(═O)H, —C(═NOR²)H, —C(═O)OH, —C(═O)OR¹, —C(═W)N(R¹R²), —CR³R⁴—N(R¹R²), —CR³R⁴—OR¹, —C(═NR¹)R³, —C(═O)R³, —CR³R⁴—C(═O)OH, —CR³R⁴—C(═O)R¹, —CR³R⁴—C(═W)N(R¹R²), —O—CR³R⁴—C(═O)OH, —O—CR³R⁴—C(═O)R¹, —O—CR³R⁴C(═W)N(R¹R²), —CR³R⁴—N(R²)—C(═W)R¹, —CR³R⁴—S(═O)₂R¹, or —CR³R⁴—N(R²)—S(═O)₂R¹; wherein
W is O or S;
R² is hydrogen, C₁-C₆-alkyl, C₂-C₆-alkenyl, C₂-C₆-alkynyl, C₁-C₆-alkoxy, C₃-C₁₁-cycloalkyl, —C(═O)—C₁-C₆-alkyl, —C(═O)—C₃-C₁₁-cycloalkyl, or —C(═O)—O—C₁-C₆-alkyl; and wherein any of the aliphatic or cyclic groups in R² are unsubstituted or substituted with 1, 2, 3, or up to the maximum possible number of identical or different radicals selected from the group consisting of halogen, hydroxy, oxo, cyano, C₁-C₆-alkyl, C₁-C₆-alkoxy, and C₃-C₁₁-cycloalkyl;
R¹ is C₁-C₆-alkyl, C₁-C₆-alkoxy, C₃-C₁₁-cycloalkyl, C₃-C₈-cycloalkenyl, C₂-C₆-alkenyl, C₂-C₆-alkynyl, C₁-C₆-alkoxyimino-C₁-C₄-alkyl, C₂-C₆-alkenyloxyimino-C₁-C₄-alkyl, C₂-C₆-alkynyloxyimino-C₁-C₄-alkyl, C₁-C₆-alkylamino, diC₁-C₆-alkylamino, —C(═O)—C₁-C₆-alkyl, —C(═O)—O—C₁-C₆-alkyl, phenyl-C₁-C₄-alkyl, phenyl-C₁-C₄-alkenyl, phenyl-C₁-C₄-alkynyl, heteroaryl-C₁-C₄-alkyl, phenyl, naphthyl, or a 3- to 10-membered saturated, partially unsaturated or aromatic mono- or bicyclic heterocycle, wherein the ring member atoms of said mono- or bicyclic heterocycle include besides carbon atoms further 1, 2, 3 or 4 heteroatoms selected from N, O and S as ring member atoms with the provision that the heterocycle cannot contain 2 contiguous atoms selected from O and S; and
wherein the heteroaryl group in the group heteroaryl-C₁-C₄-alkyl is a 5- or 6-membered aromatic heterocycle, wherein the ring member atoms of the heterocyclic ring include besides carbon atoms 1, 2, 3 or 4 heteroatoms selected from N, O, and S as ring member atoms with the provision that the heterocycle cannot contain 2 contiguous atoms selected from O and S; and wherein any of the above-mentioned aliphatic or cyclic groups are unsubstituted or substituted with 1, 2, 3, or up to the maximum possible number of identical or different groups R^{1a}; or
R¹ and R², together with the nitrogen atom to which they are attached, form a saturated or partially unsaturated mono- or bicyclic 3- to 10-membered heterocycle, wherein the heterocycle includes beside one nitrogen atom and one or more carbon atoms no further heteroatoms or 1, 2, or 3 further heteroatoms independently selected from N, O, and S as ring member atoms with the provision that the heterocycle cannot contain 2 contiguous atoms selected from O and S; and wherein the heterocycle is unsubstituted or substituted with 1, 2, 3, 4, or up to the maximum possible number of identical or different groups R^{1a}; wherein
R^{1a} is halogen, oxo, cyano, NO₂, OH, SH, NH₂, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, —NHSO₂—C₁-C₄-alkyl, —C(═O)—C₁-C₄-alkyl, —C(═O)—O—C₁-C₄-alkyl, C₁-C₆-alkylsulfonyl, hydroxyC₁-C₄-alkyl, —C(═O)—NH₂, —C(═O)—NH(C₁-C₄-alkyl), C₁-C₄-alkylthio-C₁-C₄-alkyl, aminoC₁-C₄-alkyl, C₁-C₄-alkylamino-C₁-C₄-alkyl, diC₁-C₄-alkylamino-C₁-C₄-alkyl, aminocarbonyl-C₁-C₄-alkyl, or C₁-C₄-alkoxy-C₁-C₄-alkyl;
R³, R⁴ independently of each other are selected from the group consisting of hydrogen, halogen, cyano, C₁-C₄-alkyl, C₁-C₄-alkenyl, C₁-C₄-alkynyl, C₁-C₄-haloalkyl and C₁-C₄-alkoxy; or
R³ and R⁴ together with the carbon atom to which they are bound form a cyclopropyl group,
by reacting a nitrile of formula II, wherein the variables A and R are as defined above for compounds of formula I,
N≡C-A-R II
with free hydroxylamine in a solvent, the process comprising:
step 1: charging a reaction vessel with a solvent and nitrile II;
step 2: metering 0.9 to 3.0 equivalents of free hydroxylamine, based on the amount of nitrile II, into the mixture obtained in step 1;
wherein the free hydroxylamine in step 2 is metered to nitrile II over a period of equal to or more than 15 minutes.

2. The process according to claim 1, wherein the solvent comprises an alkyl alcohol of formula III,
C₁-C₆-alkyl-OH III.

3. The process according to claim 1, wherein the solvent comprises ethanol or methanol.

4. The process according to claim 1, wherein a temperature of the reaction mixture in step 2 of the process is between 0° C. and 80° C.

5. The process according to claim 1, wherein 0.9 to 1.5 equivalents of the free hydroxylamine are added in step 2 to the reaction mixture obtained in step 1.

6. The process according to claim 1, wherein the reaction mixture obtained in step 1 contains equal to or more than 350 g of nitrile II per kilogram of solvent.

7. The process according to claim 1, wherein the free hydroxylamine in step 2 is metered to nitrile II over a period of equal to or more than 1 hour.

8. The process according to claim 1, wherein the nitrile compound is of formula II.b, 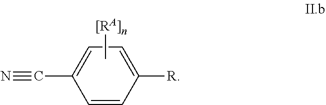
wherein n is 0 or 1, and the meaning of R^{A} and R is as defined in claim 1 for compounds of formula I, to obtain an amidoxime of formula I.b, 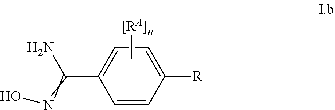
wherein the variables n, R^{A}, and R have the meaning as defined for compounds II.b.

9. The process according to claim 8, wherein the variables have the following meaning:
R^{A} is fluorine;
n is 0 or 1;
R is methyl, —C(═O)OH, —C(═O)NR¹R², —CH₂—N(R²)—C(═O)R¹, —CH₂—N(R²)—S(═O)₂R¹, 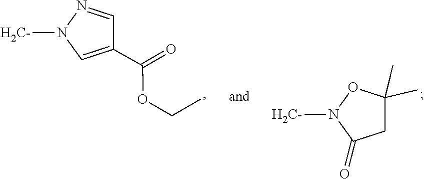
R¹ is C₁-C₆-alkyl, phenyl, or cyclopropyl, wherein the phenyl ring is unsubstituted or substituted with 1, 2, 3, or 4 identical or different groups selected from halogen;
R² is hydrogen, methyl, ethyl, methoxy, ethoxy, or cyclopropyl.

10. The process according to claim 8, wherein the variables have the following meaning:
n is 0;
R is —C(═O)N(R¹R²);
R¹ is methyl, 2-methoxyiminoethyl, bicyclo[1.1.1]pentan-1-yl, 2-fluoro-phenyl, 4-fluoro-phenyl, or 2,4-difluorophenyl; in particular methyl or 2-fluoro-phenyl;
R² is hydrogen.

11. The process according to claim 8, further comprising a step of reacting the compound of formula I.b, to obtain a compound of formula IV,

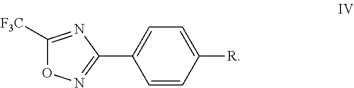

12. The process according to claim 11, wherein n is 0 and R is methyl in the compound of formula IV and further comprising a step of reacting a compound of formula IV to obtain the compound of formula IV.a.

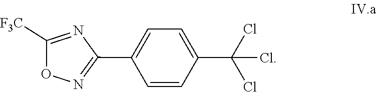 [SMILES: FC(F)(F)c1nc(-c2ccc(C(Cl)(Cl)Cl)cc2)no1]

13. The process according to claim 8, wherein R is —C(═O)OH in the compound of formula II.b and II.b, and further comprising a step of reacting the compound of formula IV to obtain the compound of formula V,

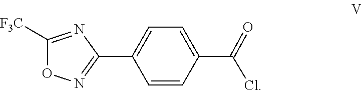 [SMILES: O=C(Cl)c1ccc(-c2noc(C(F)(F)F)n2)cc1]

14. The process according to claim 12, further comprising a step of reacting the compound of formula IV.a to obtain a compound of formula V,

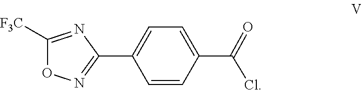 [SMILES: O=C(Cl)c1ccc(-c2noc(C(F)(F)F)n2)cc1]

15. The process according to claim 13, further comprising a step of reacting the compound of formula V with a compound of formula VI,
R¹—NH—R² VI
wherein R¹ and R² in the compound of formula VI is defined as for compounds of formula I in claim 1 to obtain a compound of formula VII,

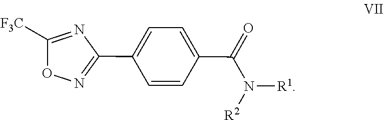

16. The process according to claim 15, further comprising a step of reacting the compound of formula V to obtain a compound of formula VIII,

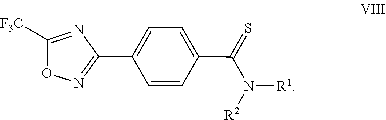

17. The process according to claim 15, wherein in the compound of formula VI
R¹ is methyl, 2-methoxyiminoethyl, bicyclo[1.1.1]pentan-1-yl, 2-fluoro-phenyl, 4-fluoro-phenyl, or 2,4-difluorophenyl; in particular methyl or 2-fluoro-phenyl; and
R² is hydrogen.
